(19) United States
(12) Patent Application Publication
(10) Pub. No.: US 2025/0228569 A1
(43) Pub. Date: Jul. 17, 2025

(54) THERAPEUTIC COMPOSITION DELIVERY DEVICE

(71) Applicant: Helios Cardio Inc., Weston, MA (US)

(72) Inventors: Kevin Cornwell, Holliston, MA (US); Peter Jackson, Wellesley, MA (US); Robert Saeid Farivar, Boise, ID (US); Benedict Taylor, Boise, ID (US); Yiannis Monovoukas, Weston, MA (US)

(73) Assignee: Helios Cardio Inc., Weston, MA (US)

(21) Appl. No.: 18/849,162

(22) PCT Filed: Mar. 22, 2023
(86) PCT No.: PCT/US2023/015879

§ 371 Date: Sep. 20, 2024

Related U.S. Application Data
(60) Provisional application No. 63/322,931, filed on Mar. 23, 2022.

(51) Int. Cl. A61B 17/122; A61B 17/00

(52) U.S. Cl. CPC A61B 17/122; A61B 2017/00778; A61B 2017/00893

(57) ABSTRACT

A clip, such as an atrial clip, includes a first arm, a second arm, and a hinge rotatably coupling the first arm and the second arm. The first arm and the second arm are selectively moveable between an open configuration and a closed configuration. In the closed configuration, the first arm and the second arm engage with and clamp a piece of cardiac tissue between them. The atrial clip also includes a lock, which retains the first arm and the second arm in the closed configuration, as well as at least one porous material attached to one or both of the arms. The porous material is configured to absorb and elute a therapeutic composition to the cardiac tissue, wherein the therapeutic composition is configured to prevent and/or treat postoperative atrial fibrillation.

### CROSS-REFERENCE TO RELATED APPLICATIONS

This application claims priority under 35 U.S.C. § 119 (e) to U.S. Provisional Application Ser. No. 63/322,931, filed Mar. 23, 2022, and entitled “THERAPEUTIC COMPOSITION DELIVERY DEVICE,” which is incorporated herein by reference in it its entirety and for all purposes.

### BACKGROUND

Clips may be used to prevent a flow of blood from entering or circulating through a portion of a circulatory system, including a heart. This includes atrial clips which may be applied to an atrial appendage of a subject's heart to prevent the circulation of blood in the now occluded atrial appendage.

### SUMMARY

In some embodiments, a clip may comprise a first arm, a second arm, and a hinge rotatably coupling the first arm and the second arm. The first arm and the second arm may be selectively movable between an open configuration and a closed configuration. The first arm and the second arm may be configured to engage with and clamp cardiac tissue therebetween in the closed configuration. The clip may further comprise a lock configured to retain the first arm and the second arm in the closed position. The clip may further comprise at least one piece of porous material attached to at least one of the first arm and the second arm. The at least one piece of porous material may be configured to receive and deliver a therapeutic composition.

In some embodiments, a method of delivering a therapeutic composition to a heart of a subject may comprise attaching a clip to a first cardiac tissue. The clip may include a porous material containing the therapeutic composition. The method may further comprise delivering the therapeutic composition from the porous material to the heart of the subject.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a schematic of a heart;

FIG. 1B is a close-up view of the schematic of FIG. 1A depicting an atrial appendage;

FIG. 1C is a schematic of one embodiment of an atrial clip according to the present disclosure attached to the atrial appendage of FIG. 1B;

FIG. 1D is a schematic of one embodiment of an atrial clip according to the present disclosure attached to the atrial appendage of FIG. 1B;

FIG. 2A is a schematic of one embodiment of an atrial clip in an open configuration;

FIG. 2B is a schematic view of the embodiment of FIG. 2A in a closed configuration;

FIG. 2C is a cross-sectional schematic of the embodiment of FIG. 2A in a closed configuration;

FIG. 3A is a schematic of one embodiment of an atrial clip in an open configuration;

FIG. 3B is a cross-sectional schematic view of the embodiment of FIG. 3A in a closed configuration;

FIG. 4A is a schematic of one embodiment of an atrial clip in an open configuration;

FIG. 4B is a cross-sectional schematic view of the embodiment of FIG. 4A in a closed configuration;

FIG. 5A is a schematic of one embodiment of an atrial clip in an open configuration;

FIG. 5B is a cross-sectional schematic view of the embodiment of FIG. 5A in a closed configuration;

FIG. 6A is a schematic of a kit including a clip with an attached porous material and a therapeutic composition, according to an embodiment;

FIG. 6B is a cross-sectional schematic view of an embodiment of an atrial clip having a porous material including a barrier layer, according to an embodiment;

FIG. 6C is a cross-sectional schematic view of an embodiment of an atrial clip having a barrier layer disposed between two layers of porous material, according to an embodiment;

FIG. 7A is an image of a porous material during a step of soaking the porous material in a therapeutic composition, according to an embodiment;

FIG. 7B is an image of the porous material of FIG. 7A after the soaking step, with the porous material saturated with the therapeutic composition; and

FIG. 8 is a method for delivering a therapeutic composition to heart tissue, according to an embodiment.

### DETAILED DESCRIPTION

Open heart cardiovascular procedures, such as coronary artery bypass grafting (CABG), valve replacement, or ventricular assist devices (VAD) implant procedures, are associated with a high rate of postoperative complications. Of those complications, postoperative atrial fibrillation (POAF) is extremely common, occurring in 30-50% of patients depending on the procedure and patient comorbidities. First onset of POAF occurs in a non-linear fashion, with a peak likelihood of incidence 2 to 3 days after surgery, and then decreases with time. POAF is associated with an increased risk of stroke and a 10-year increased risk of cardiovascular and all-cause mortality. Treatment for POAF may also result in significant additional costs for the patient.

Risk of POAF may be reduced by closing off an atrial appendage during a procedure. Atrial appendage closure procedures may involve occluding a flow of blood to an atrial appendage in order to prevent stagnation and clotting of blood within or around the atrial appendage. Occlusion of an atrial appendage may be accomplished by plugging the atrial appendage internally, by cinching a band or ligature around the outside of the atrial appendage, or by clamping an atrial clip to pinch the atrial appendage closed.

Risk of POAF may also be reduced using drugs or therapeutic compositions. An example of a therapeutic composition prescribed for preventing or treating POAF is amiodarone, a Class III antiarrhythmic drug that blocks voltage-gated potassium channels leading to prolonged repolarization of the cardiac action potential. Given orally, amiodarone has a slow onset of action. To achieve therapeutic concentrations of amiodarone in the myocardium, high oral doses of the drug must be given daily to the patient over a prolonged period of time. Systemic delivery (e.g., intravenous or oral) is associated with severe side effects on the thyroid, liver, lungs, skin and eyes. Local delivery of amiodarone has been investigated using several methodologies, including direct drug infusions into the pericardial fluid via a pump, and applications directly to the epicardium of the heart via spray-on gels or thin films, or with epicardial patches. However, these procedures and methods have been unable to deliver large enough concentrations over sufficiently long durations to provide clinically relevant therapeutic benefits either due to their loading capacity being too low and/or their delivery kinetics not being well matched for the desired dosing durations.

In view of the above, the inventors have recognized the benefits associated with an atrial clip which includes a porous material attached thereto capable of occluding blood flow to an atrial appendage while delivering relatively large dosages of a therapeutic composition over longer durations of time as compared to prior therapeutic devices and procedures. Accordingly, embodiments related to an atrial clip including a porous material with an appropriate combination of mechanical and material parameters may be provided in some embodiments. Such an atrial clip may thus be configured for both occluding blood flow to an atrial appendage and delivering a desired therapeutic composition to cardiac tissue as described herein.

In some embodiments, a clip may comprise a first arm, a second arm, and a hinge rotatably coupling the first arm and the second arm. The first arm and the second arm may be selectively movable between an open configuration and a closed configuration. The first arm and the second arm may be configured to engage with and clamp cardiac tissue therebetween in the closed configuration. The first arm and the second arm may be configured to engage with and clamp any appropriate cardiac or other type of tissue. In certain embodiments, the first arm and the second arm may be configured to engage with and clamp an atrial appendage therebetween in a closed configuration in order to occlude a flow of blood through the atrial appendage. In some instances a clip may comprise a lock configured to retain the first arm and the second arm in the closed position. The clip may further comprise at least one piece of porous material attached to at least one of the first arm and the second arm. The porous material may be configured to absorb a desired loading of one or more therapeutic compounds that may be eluted from the porous material over a desired time period. For example, the one or more therapeutic compounds may be eluted into the pericardial space surrounding the heart of a subject in some applications.

In some embodiments, a method of delivering a therapeutic composition to a heart of a subject may comprise attaching a clip, such as an atrial clip, to a first cardiac tissue, such as an atrial appendage of the heart. The clip may include a porous material containing the therapeutic composition. Additionally, the method may include delivering the therapeutic composition from the porous material to the heart of the subject while the clip is attached to the cardiac tissue. For example, in some instances the therapeutic composition may be eluted from the porous material into a pericardial space surrounding the heart of a subject such that the therapeutic composition may be delivered to regions of the heart that are distanced from the clip.

In some embodiments, a clip with a porous material may be attached to any appropriate cardiac tissue including, in one embodiment, left atrial appendage or a right atrial appendage to deliver a therapeutic composition to one or more desired cardiac tissues. In other embodiments, a clip with porous material may be attached at other locations, including the pericardial sac. Thus, a clip according to the present disclosure may be any clip capable of being attached to any desired type of cardiac tissue.

When attached to a desired cardiac tissue, a clip including a porous material loaded with a therapeutic composition may elute the therapeutic composition contained within the porous material such that the therapeutic composition is delivered to the cardiac tissue to which the porous material is attached. Additionally or alternatively, the therapeutic composition may be delivered to a second cardiac tissue spaced apart from the first cardiac tissue to which the porous material is attached. For example, in one specific embodiment, a clip including a porous material may be attached to the atrial appendage of a subject and the therapeutic composition may diffuse out of the porous material into the space surrounding the heart within the pericardial sac such that it delivers a therapeutically relevant dose to the myocardium, or other cardiac tissue, of the subject at locations both adjacent to and removed from the attachment location of the clip.

In view of the above, the inventors have recognized and appreciated designs for an atrial clip and therapeutic composition delivery device that engages with and clamps cardiac tissue while also locally delivering a therapeutic composition to the cardiac tissue for the prevention of POAF. In some embodiments, the device may occlude blood flow to an atrial appendage by engaging with and clamping the atrial appendage. The therapeutic compositions may be applied to the porous material of the atrial clip in the operating room during heart surgery and/or may be applied during manufacturing of the porous material or any time prior to surgery. In some embodiments, the device may use a specially configured version of a biopolymer scaffold material, for example EBM, which may act as a physical barrier to inflammatory mediastinal constituents while effectively providing a reservoir for a therapeutic composition. Of course, while the devices are described below with respect to treatment and prevention of POAF, it should be noted that the disclosure is not so limited, and the device may be used in different applications in the body.

In some embodiments, a length of an atrial clip or an arm thereof may be greater than or equal to 2 cm, 4 cm, 6 cm, and/or any other appropriate length. Additionally, the length may be less than or equal to 7 cm, 5 cm, 3 cm, and/or any other appropriate length. Combinations of the foregoing are contemplated including, for example, greater than or equal to 2 cm and less than or equal to 7 cm, greater than or equal to 4 cm and less than or equal to 6 cm, and/or any other appropriate combination of the foregoing. Of course, while particular ranges for the length of an atrial clip or an arm thereof are provided above, it should be understood that other ranges both greater than and less than those noted above are also contemplated as the disclosure is not limited in this fashion.

In some embodiments, a width of an atrial clip or an arm thereof may be greater than or equal to 0.5 cm, 0.75 cm, 1.25 cm, and/or any other appropriate width. Additionally, the width may be less than or equal to 2 cm, 1.5 cm, 1 cm, and/or any other appropriate width. Combinations of the foregoing are contemplated including, for example, greater than or equal to 0.5 cm and less than or equal to 2 cm, greater than or equal to 1 cm and less than or equal to 1.5 cm, and/or any other appropriate combination of the foregoing. Of course, while particular ranges for the width of an atrial clip or an arm thereof are provided above, it should be understood that other ranges both greater than and less than those noted above are also contemplated as the disclosure is not limited in this fashion.

Appropriate porous materials may include biopolymer scaffold materials, collagen foams, or permanent or degradable synthetic or natural polymers. In one specific embodiment, the porous material may be a biopolymer scaffold material such as bovine extracellular matrix (EBM) which may be loaded with a desired therapeutic composition for delivery to a target heart tissue as described herein. The EBM, or other appropriate biopolymer scaffold or porous material, may be decellularized in some embodiments to remove the allogenic and/or xenogeneic cellular antigens from the scaffold to reduce and/or prevent an immune response to the scaffold. Other specific examples of porous materials that may be used with the disclosed embodiments, may include, but are not limited to permanent or degradable synthetic or natural polymers, biopolymer scaffold materials, and collagen foams.

In some embodiments, the EBM or other biopolymer scaffold material may be made using fetal (i.e., prebirth) or neonatal bovine dermis that is less than 10 weeks of age post birth, 26 weeks of age post birth, and/or 52 weeks of age post birth. Without wishing to be bound by theory, selecting tissue from animals of different ages within the above ranges may permit the formation of different porous materials with different thicknesses, porosity, collagen content, and/or other desirable parameters. Additionally, in some embodiments, the therapeutic composition used with the porous material may be a therapeutic composition for treating and/or preventing post-operative atrial fibrillation such as amiodarone.

The characteristics of certain porous materials described herein make these materials especially suited for delivering therapeutic compositions to the heart when used as a component of an atrial clip. However, it should be noted that the porous material may be made of any material that provides a desired combination of properties, including permanent or degradable synthetic or natural polymers, biopolymer scaffold materials, and collagen foams. EBM as described herein is one example of a porous material that has a desired combination of thickness, porosity, pore size, tortuosity, collagen fiber architecture, mechanical strength, biochemistry, surface properties, and/or other appropriate material parameters that make it well-suited as a porous material in the atrial clip described herein. However, other materials are also contemplated which provide the same, similar, or different combinations of properties for various applications.

Generally, EBM is a bioremodelable, biopolymer scaffold material derived from fetal, neonatal or post-natal animal tissue. EBM is processed in a way that preserves its tissue strength without reducing its intrinsic biological properties or compromising the ability of cells that occupy the tissue to remodel it. EBM may be used as a tissue-building component with or without cells for creating human body replacements.

According to some embodiments, the EBM, biopolymer scaffold material, or other porous material may be produced from animal tissue by a method comprising the following steps: (1) removing the tissue from its source; (2) removing undesired cells, proteins, lipids, nucleic acids, and carbohydrates via chemical methods such as sodium chloride, hydrogen peroxide, sodium hydroxide, water and other optional solvents or chemicals; optionally extracting growth and differentiation factors from the tissue; (3) inactivating infective agents of the tissue; (4) mechanically expressing undesirable components from the tissue; (5) washing the tissue for removal of chemical residues; (6) optionally drying via lyophilization, supercritical CO2, air-drying, or other method; and (7) optionally cross-linking the tissue after chemical and mechanical treatment; and (8) optionally terminally sterilizing. As noted above, in some embodiments, a porous material such as EBM may be made using fetal or neonatal bovine tissue that is any appropriate age that is less than 52 weeks of age, though embodiments in which tissue from an older animal is used is also contemplated. These manufacturing processes are further described in U.S. Pat. No. 9,011,895 which is incorporated herein by reference in its entirety for all purposes.

In some applications, it may be desirable to provide a particular collagen fiber architecture that is close to the collagen fiber architecture of the cardiac tissue that a porous material is applied to. Accordingly, in some embodiments, the porous material may include the following ranges and types of collagens. In some embodiments, the collagen contained in the matrix may be a native, intact, and/or non-denatured collagen from the original base matrix material. A porous material, such as EBM or other biopolymer scaffold material, may include type I collagen in a dry weight percentage relative to the overall weight of the porous material that is greater than or equal to 60 wt %, 70 wt %, 75 wt %, 80 wt %, 90 wt % and/or any other appropriate range. The weight percentage of the type I collagen may also be less than or equal to 96%, 95%, 90 wt %, 85 wt %, 80 wt %, and/or any other appropriate range. The porous material may also include a large quantity of type III collagen in a dry weight percentage relative to the overall weight of the porous material that is greater than or equal to 4 wt %, 5 wt %, 10 wt %, 15 wt %, 20 wt %, and/or any other appropriate range. Correspondingly, the weight percentage of type III collagen may be less than or equal to 40 wt %, 30 wt %, 25 wt %, 20 wt %, 10 wt %, and/or another appropriate range. Combinations of the above are contemplated including, for example, type I collagen in a range between or equal to 60 wt % and 95 wt % and type III collagen in a range between or equal to 5 wt % and 40 wt %, or more preferably 60 wt % to 80 wt % type I collagen as well as 20 wt % to 40 wt % type III collagen, may correspond to material formed from fetal and neonatal tissue. Correspondingly, type I collagen in a range around about 95 wt % and type III collagen around about 5 wt % may correspond to adult dermis. This difference in collagen type content may lead to different material properties. Of course, the inclusion of other types of collagens as well as weight percentages different than those noted above are also contemplated as the disclosure is not limited in this fashion.

EBM may be a suitable porous material at least in part because, since the manufacturing process does not significantly damage the native collagen fibers physically or biochemically, EBM may not incite significant inflammation. EBM may also be substantially free of xenogeneic growth factors that incite inflammation. In applications where EBM is used to provide a therapeutic composition to cardiac tissue, EBM may not damage, injure, or otherwise further exacerbate any trauma to the cardiac because it may be placed on the epicardium and/or pericardium in some embodiments rather than placed directly on the myocardial tissue. The EBM may also be devoid of measurable quantities of xenogeneic growth factors or extracellular matrix proteins that may cause inflammation which other porous materials may retain.

Benefits associated with increased thicknesses of the porous materials disclosed herein may include, for example, increased reservoir volumes for containing a therapeutic composition for long-duration release. Thus, in some embodiments, the porous material may have a thickness that is greater than or equal to about 0.2 mm, 0.5 mm, 1 mm, 2 mm, 3 mm, 4 mm, 5 mm, and/or any other appropriate thickness. Additionally, the thickness of the porous material may be less than or equal to 6 mm, 5 mm, 4 mm, 3 mm, 1.5 mm, and/or any other appropriate thickness. Combinations of these thicknesses are contemplated including, for example, a thickness that is between or equal to 0.5 mm and 6 mm. Of course, thicknesses both greater than and less than those noted above are also possible.

Other benefits of EBM or other biopolymer scaffold materials include their absorption rates and absorption capacities. An EBM or other biopolymer scaffold material may be lyophilized, resulting in a highly porous membrane that can rapidly absorb large quantities of therapeutic compositions, which in some embodiments may be suspended in a carrier, even when provided in thickness greater than 1 mm thick configuration. Furthermore, EBM or another biopolymer scaffold material may be lyophilized at the end of manufacture, which, in combination with the thickness range of the device, may lead to a device with extremely high liquid absorption characteristics. Thus, in some embodiments, a porous material, such as EBM or other biopolymer scaffold matrix, may have an areal absorption capacity that is greater than or equal to 0.1 ml/cm², 0.2 ml/cm², 0.3 ml/cm², 0.4 ml/cm², 0.5 ml/cm², and/or any other appropriate absorption capacity (herein areal absorption capacity means volume of fluid per square area of porous material, however effective absorption capacity means volume of fluid per volume of porous material) and, depending on the therapeutic composition, may be saturated in less than 10 minutes. The areal absorption capacity may also be less than or equal to 1.0 ml/cm², 0.9 ml/cm², 0.8 ml/cm², 0.7 ml/cm², 0.6 ml/cm², and/or any other appropriate absorption capacity. Combinations of these ranges are contemplated including, for example, an areal absorption capacity that is between or equal to 0.1 ml/cm² and 1.0 ml/cm².

Depending on the mass of a given therapeutic composition, and the concentration of the therapeutic composition within a liquid absorbed into the porous material, the therapeutic composition may be present in a range of areal densities. For example, a therapeutic composition, such as amiodarone in some embodiments, may be disposed in the pores of a porous material (e.g., EBM or other porous material), may have an areal density greater than or equal to 5 mg/cm², 6 mg/cm², 7 mg/cm², 10 mg/cm², and/or any other appropriate density. The areal density of the therapeutic composition may also be less than or equal to 30 mg/cm², 20 mg/cm², 10 mg/cm², 9 mg/cm², 8 mg/cm², 7 mg/cm², 6 mg/cm², and/or any other appropriate areal density. Combinations of the foregoing are contemplated including, for example, an areal density of a therapeutic composition contained within a porous material that is between or equal to 5 mg/cm² and 10 mg/cm². In one specific example, amiodarone dissolved in a liquid at a concentration of 50 mg/mL might result in 1-5 mg/cm² of amiodarone loaded in the porous material. Of course, it should be understood that other ranges both greater than and less than those noted above are also contemplated.

In order to provide a desired amount of therapeutic composition to a subject, a desired amount of a therapeutic composition may be absorbed into the porous material for a given application. For example, in some embodiments, an amount of amiodarone absorbed into a porous material may be greater than or equal to 1 mg, 100 mg, 200 mg, 300 mg, 400 mg, 500 mg, 1000 mg, 2000 mg, and/or any other appropriate amount. Amounts of amiodarone absorbed into the porous material may also be less than or equal to 3000 mg, 2000 mg, 1000 mg, 500 mg, and/or any other appropriate amount. Combinations of the foregoing are contemplated including, for example, an amount of amiodarone that is absorbed into a porous material that is between or equal to 1 mg and 3000 mg. Of course, other combinations of the foregoing as well as ranges both greater than and less than the ranges noted above are also contemplated as the disclosure is not so limited.

While a porous material may be provided pre-soaked with a desired therapeutic composition in a carrier liquid in some embodiments, in other embodiments, a user may soak the porous material with the desired therapeutic composition prior to implantation. Due to the highly porous absorbent nature of the materials disclosed herein, the porous material may be configured to absorb a desired amount of a therapeutic composition corresponding to the loadings disclosed herein in a time period that is less than or equal to 10 minutes, 5 minutes, 3 minutes, 2 minutes, 1 minute, and/or any other appropriate time period.

Porous materials as described herein may be provided with a range of surface areas and/or shapes for various applications including any of the applications described previously above. Depending on the specific application, appropriate sizes for the porous material may include surface areas that are greater than or equal to 1 cm², 20 cm², 50 cm², and 100 cm², 150 cm², 200 cm², and/or any other appropriate size. The size of the porous material may also include surface areas that are less than or equal to 200 cm², 150 cm², 100 cm², 50 cm², 20 cm², 1 cm², and/or any other appropriate size. Combinations of the foregoing are contemplated including, for example, porous material with surface areas that are between or equal to 0.1 cm² and 200 cm².

Dimensions for a porous material may be selected to achieve a desired size or area. Depending on the application, an appropriate length for a porous material may be greater than or equal to 1 cm, 3 cm, 5 cm, 7 cm, or any other appropriate length. Additionally or alternatively, the length for a porous material may be less than or equal to 10 cm, 7 cm, 5 cm, or any other appropriate length. Combinations of the foregoing are contemplated including, for example, porous material with lengths that are between or equal to 1 cm and 10 cm.

Additionally, depending on the application, an appropriate height for a porous material may be a coating on the order of microns or millimeters, or a height may be greater than or equal to 1 cm, 3 cm, 5 cm, 7 cm, or any other appropriate height. Additionally or alternatively, the height for a porous material may be less than or equal to 10 cm, 7 cm, 5 cm, or any other appropriate height. Combinations of the foregoing are contemplated including, for example, porous material with heights that are between or equal to 1 cm and 10 cm.

EBM also has superior mechanical properties as compared to other typical porous materials. For example, it is a strong, yet elastic material. By selecting for age, without splitting, and by nature of the collagen fiber architecture unique to the bovine source, EBM is extremely strong yet remains soft and pliable with stiffness similar to other human soft tissues (unlike most synthetic polymers, metals, or ceramics) including pericardium and myocardium. Thus, EBM based devices are mechanically strong (stronger than other scaffold materials such as SIS). Additionally, EBM based devices may be compliant and soft such that they do not significantly affect heart function or result in mechanical rubbing and/or abrading of the myocardium when contacting the heart during normal sinus rhythm.

In view of the above, a porous material as disclosed herein, including an EBM or other porous material, may have an ultimate tensile strength that is greater than or equal to 3 MPa, 5 MPa, 10 MPa, 20 MPa, 30 MPa, 40 MPa, and/or any other appropriate tensile strength. The ultimate tensile strength may also be less than or equal to 60 MPa, 50 MPa, 40 MPa, 30 MPa, and/or any other appropriate tensile strength. Combinations of foregoing are contemplated including, for example, an ultimate tensile strength of a porous material which may be between or equal to 3 MPa and 60 MPa. Of course, tensile strengths both greater than and less than those noted above are also contemplated as the disclosure is not so limited.

A porous material, such as an EBM or other porous material, may also have an improved suture pullout as compared to other materials. For example, a suture retention strength of the porous material may be greater than or equal to 10 N, 20 N, 50 N, 100 N, 200 N, 300 N, and/or any other appropriate suture retention strength. The suture retention strength of the porous material may also be less than or equal to 500 N, 400 N, 300 N, 200 N, 100 N, and/or any other appropriate suture retention strength. Combinations of the foregoing are contemplated including, for example, a suture retention strength that is between or equal to 10 N and 500 N. Of course, suture retention strengths both greater than and less than those noted above are also contemplated as the disclosure is not limited in this fashion. The above-noted suture retention strengths may be measured using a suture having a thickness equivalent to USP suture size/diameter for the intended procedure (4-0-2) during a standard suture pull out test.

In some instances, and as noted above, it may be desirable for a porous material, such as EBM or other biopolymer scaffold material, to exhibit a desired amount of elasticity for cardiac applications. Accordingly, in some embodiments, a Young's modulus, sometimes referred to as an elastic modulus, of the porous material may be greater than or equal to 3 MPa, 10 MPa, 20 MPa, 30 MPa, 40 MPa, 50 MPa, 100 MPa, 200 MPa, and/or any other appropriate range. The Young's modulus may also be less than or equal to 400 MPa, 300 MPa, 200 MPa, 100 MPa, 50 MPa, 40 MPa, 30 MPa, and/or any other appropriate range. Combinations of the foregoing are contemplated including, for example, between or equal to 1 MPa and 400 MPa as well as between or equal to 20 MPa and 200 MPa. Of course, while specific ranges are provided, ranges both greater than and less than those noted above are also contemplated as the disclosure is not limited in this fashion.

Another benefit of EBM and other biopolymer scaffold materials is the tortuous interconnected open pore structure of the material. Thus, a porous material, such as EBM or other porous material, may include a plurality of interconnected open pores that connect a first surface of the to an interior portion of the porous material, and in some embodiments, a second opposing surface of the porous material. A flow path extending through the plurality of interconnected pores may follow a tortuous, i.e., non-linear, path such that a liquid may flow into and subsequently out of the porous material during loading and eluting phases of the material. In some examples, the interconnected tortuous porosity of a porous material is provided by a crisscrossing collagen fiber architecture of the porous material. This porosity and architecture allow fluid to be absorbed quickly in a surgical setting (e.g., minutes) while still acting as a barrier against inflammation inducing molecules and/or providing the desired therapeutic composition elution properties.

A porous material used in the embodiments disclosed herein may have a porosity that is greater than or equal to 20%, 30%, 40%, 50%, and/or other appropriate porosity. The porosity may also be less than or equal to 80%, 70%, 60%, 50%, and/or any other appropriate porosity. Combinations of foregoing are contemplated including, porosities that are between or equal to 20% and 80%. However, porosities both greater than and less than those noted above are also contemplated as the disclosure is not so limited. Additionally, without wishing to be bound by theory, the high porosity nature of the materials disclosed herein may affect the overall volume and areal capacities of the materials.

A porous material, such as EBM or other porous material, used in the embodiments disclosed herein may have an average pore size that is greater than or equal to 1 μm, 2 μm, 5 μm, 10 μm, 50 μm, 100 μm, 250 μm, and/or any other appropriate size. The average pore size may also be less than or equal to 500 μm, 250 μm, 100 μm, 50 μm, and/or any other appropriate size. Combinations of foregoing are contemplated including, for example, an average pore size of a porous material that is between or equal to 1 μm and 500 μm. Of course, average pore sizes both greater than and less than those noted above are also contemplated. Without wishing to be bound by theory, the pore sizes and tortuosity of the materials disclosed herein may affect the absorption rates of a therapeutic composition into the porous material. For example, materials with larger pore sizes and decreased tortuosity, may exhibit faster rates during both absorption and elution as compared to materials exhibiting smaller pore sizes and increased tortuosity.

Due to the collagen fibers swelling and shrinking depending on the exposure of a porous material to a given liquid, the above porosities and average pore sizes may be measured in the dry state prior to introduction of a carrier liquid and/or therapeutic composition to the porous material. Additionally, the pore sizes and porosity may be measured using microscopic optical image analysis.

While many of the embodiments and properties described herein are described with respect to EBM which may exhibit advantageous properties for certain applications, it will be appreciated that EBM is provided only as an example of a single type of porous material that may be appropriate for some applications. As previously described, other materials may exhibit properties that are the same as, similar to, or different from those described above. Thus, any porous material exhibit a desired combination of properties for absorbing and subsequently eluting a therapeutic compound over time may be used in an atrial clip as disclosed herein as the disclosure is not limited in this respect.

In some embodiments, it may be desirable to improve the wicking capabilities of a porous material. Such a modification may improve the ability of the porous material to absorb liquids, may alter the elution kinetics of a therapeutic compound from the porous material, and/or may help to reduce the creation of unfilled occluded portions of the material due to the inclusion of air pockets in the matrix. Accordingly, in some embodiments, it may be desirable to include hydrophilic modifications and/or surface coatings within the pores of the porous material. In such an embodiment, the surface modification and/or coating on the surface of the pores of the porous material may be more hydrophilic, i.e., exhibit a lower water contact angle, than the underlying porous material itself. Appropriate types of hydrophilic modifications and/or coatings may include, but are not limited to: polyethylene glycol (PEG), crosslinked collagen; degradable polymers such as poly(lactic acid) (PLA), poly (glycolic acid) (PGA), and P4HB; and/or permanent polymers. Of course, embodiments in which a hydrophilic modification and/or coating are not used are also contemplated.

As described above, a porous material, such as EBM or other biopolymer scaffold material, acts as a reservoir for a therapeutic composition. When the porous material containing a therapeutic composition is attached to tissue in a subject via an associated clip, the device may locally deliver the therapeutic composition from the porous material to adjacent tissues and/or other tissues spaced apart from the porous material. Another benefit of the disclosed porous materials, including EBM or other biopolymer scaffold material, are the therapeutic composition release characteristics that allow for therapeutic composition delivery in a specified therapeutic window. The therapeutic compositions may not bind strongly to the collagen of the device in some embodiments. Also, the porosity and fluid flow characteristics of porous material slow down the release of the therapeutic composition as compared to a bolus or simple injection delivery to the pericardial fluid. Due to these properties, in some embodiments, the release and/or accumulation of a therapeutic composition in a target volume and/or tissue may increase over the first several days, resulting in a peak therapeutic concentration in a target tissue within the heart at a time between 1 day and 5 days, 2 days and 4 days, and/or more preferably at about 3 days after implantation. Without wishing to be bound by theory, providing a desired therapeutic concentration of a therapeutic composition in a time range around 3 days may be desirable for applications such as treating and/or preventing postoperative atrial fibrillation which sees the largest rate of occurrence on or around day 3. In some embodiments, a minimum elution rate and/or concentration of the therapeutic composition may continue to be delivered from the device to the target heart tissue for 28 days, 20 days, 10 days, 7 days, and/or any other appropriate time period. Since this release of the therapeutic composition is local, it may reduce the systemic load of amiodarone, or other therapeutic composition, to other organs and tissues. Delivery may also occur fast enough that the several days of pre-loading used in other surgical procedures to reach a desired therapeutic concentration may be unnecessary.

It should be understood that the desired peak concentration may be different for different therapeutic compositions. However, in the case of amiodarone, a desired peak therapeutic concentration that is between or equal to 1 μg/g and 500 μg/g 1000 in the target tissue within the noted time periods above may be desired in some embodiments. The minimum therapeutic concentration may be maintained for the time periods noted above after the peak. The minimum therapeutic concentration may be less than a corresponding peak therapeutic concentration and in some embodiments may be between or equal to 1 μg/g and 20 μg/g in the target tissue. The expected concentrations of the therapeutic composition in a target tissue may be evaluated using an appropriate animal model as detailed in the examples below.

In some embodiments, it may be desirable to provide directionality to the delivery of a therapeutic composition. For example, it may be desirable to reduce, or substantially prevent, the elution of a therapeutic composition from one surface of a porous material while permitting the therapeutic composition to elute from a second opposing surface of the porous material. In one such embodiment, it may be desirable to deliver a therapeutic composition to the heart while preventing delivery of the therapeutic composition to the sternal side of a patch made from the porous material. Appropriate barriers that may be applied to, or otherwise disposed on, a surface of the porous material may include, but are not limited to: polyethylene glycol (PEG), crosslinked collagen; degradable polymers such as poly (lactic acid) (PLA), poly (glycolic acid) (PGA), and P4HB; permanent polymers; and/or any other appropriate material capable of reducing the diffusion rate and/or completely blocking the passage of the therapeutic composition. In such an embodiment, at least a portion, and in some embodiments the entirety, of the opposing surface of the porous material may be substantially free of the barrier material such that the therapeutic composition may be eluted from the uncovered surface. Depending on the embodiment, the barrier may either be a separate layer disposed on a surface of the porous material and/or the barrier may be disposed in the pores adjacent to the surface of the porous material as the disclosure is not limited as to how the barrier layer is formed.

As used herein, the term “therapeutic composition” (also referred to interchangeably as a “drug” or “therapeutic agent”) refers to a composition that is administered to a subject to treat a disease, disorder, or other clinically recognized condition, or for prophylactic purposes, and has a clinically significant effect on the body of the subject to treat, prevent, and/or diagnose the disease, disorder, or condition. The therapeutic composition may be delivered to a subject in a quantity greater than a trace amount to affect a therapeutic response in the subject. In some embodiments, therapeutic compositions can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals. Certain such therapeutic compositions may include molecules such as proteins, peptides, hormones, nucleic acids, gene constructs, cells (e.g., autologous cells, allogeneic cells, and/or any other appropriate type of cell), etc., for use in therapeutic, diagnostic, and/or enhancement areas. In certain embodiments, the therapeutic composition is a small molecule and/or a large molecule. Accordingly, it should be understood that the therapeutic compositions described herein are not limited to any particular type of therapeutic composition.

In some embodiments, and as noted above, a therapeutic composition may be a therapeutic composition used to prevent and/or treat postoperative atrial fibrillation. Appropriate therapeutic compositions for this purpose, may include, but are not limited to, antiarrhythmic therapeutic compositions such as amiodarone; lidocaine; magnesium; salicylic acid; beta blockers; antibiotics, and/or any other appropriate therapeutic composition capable of preventing and/or treating postoperative atrial fibrillation.

To facilitate absorption of a desired therapeutic composition into a porous material, in some embodiments, a therapeutic composition may be dissolved, dispersed, and/or otherwise mixed with a carrier liquid. Appropriate types of carrier liquids may include, but are not limited to: water; saline; polysorbate; alcohol including benzyl alcohol, methanol, ethanol, or other appropriate type of alcohol; plasma; serum; other bodily fluids, and/or any other appropriate type of carrier. Of course, embodiments in which a carrier liquid is not used are also contemplated.

It should be understood that the porous materials disclosed herein may be used for any appropriate applications within the heart, and/or other portions of the body. Appropriate applications include, but are not limited to engaging with, clamping, and delivering a therapeutic composition to a cardiac tissue including a pericardial sac, a pericardial tissue, an epicardial tissue, an endocardial tissue, or a myocardial tissue. The cardiac tissue may be located at any position of the heart, including an atrial appendage or any other desired location.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

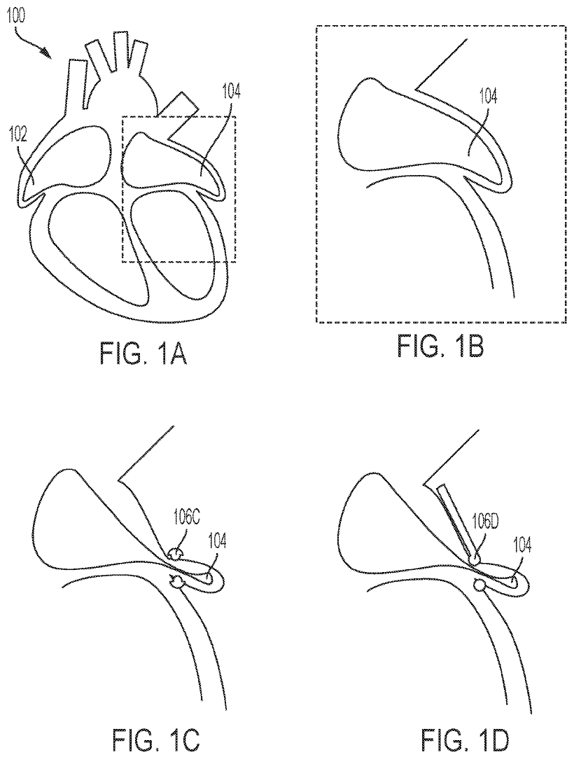

FIGS. 1A-1D depict a heart of a subject and an atrial clip attached thereto. In the embodiment shown, a heart 100 may have a first atrial appendage 102 and a second atrial appendage 104. A first embodiment of an atrial clip 106C having two pieces of porous material may be attached to the second atrial appendage 104 as shown in FIG. 1C. A second embodiment of an atrial clip 106D having only a single larger piece of porous material may be attached to the second atrial appendage 104 as shown in FIG. 1D.

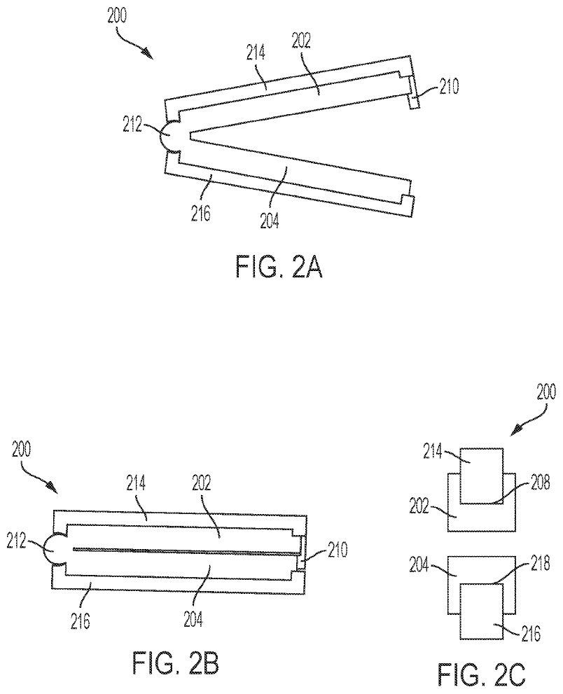

FIGS. 2A-2C show one embodiment of an atrial clip according to the present disclosure. An atrial clip 200 may include a first arm 202 and a second arm 204. A hinge 212 may rotatably couple the first arm 202 and the second arm 204, such that the first arm 202 and the second arm 204 are selectively moveable between an open configuration (shown in FIG. 2A) and a closed configuration (shown in FIG. 2B). Any appropriate hinge may be used to rotatably connect the first and second arm of the various embodiments disclosed herein including, for example, a living hinge, a pin joint, or any other appropriate rotatable connection. In the open configuration, each of the first arm 202 and the second arm 204 may be permitted to rotate about the hinge 212. In the closed configuration, each of the first arm 202 and the second arm 204 may be configured to engage with and clamp cardiac tissue disposed between the first arm and the second arm. For example, in the closed configuration, each of the first arm 202 and the second arm 204 may be configured to engage with and clamp an atrial appendage therebetween, as illustrated in FIG. 1C.

The atrial clip 200 may further include a lock 210. The lock 210 may be configured to retain the first arm 202 and the second arm 204 in the closed configuration. The lock may include any appropriate retaining or fastening structure, including mechanical fasteners such as snaps, clamps, crimps, or mechanically interlocking features.

The first arm, the second arm, the hinge, and the lock of the atrial clip may be formed from any appropriate material, including, but not limited to, plastics, polymers, or metals. Depending on the specific embodiment, these components may be formed from a medical grade polymer or a biocompatible material such as polypropylene, although it will be appreciated that any appropriate material may be used as the disclosure is not limited in this respect. These components may be formed as a single piece, or they may be formed separately and assembled together to form the atrial clip.

The atrial clip 200 may further include a porous material. In the embodiment, shown, a first piece of porous material 214 may be attached to the first arm 202, and a second piece of porous material 216 may be attached to the second arm 204. The porous material, or each piece thereof, may be configured to absorb a therapeutic composition as described herein. The porous material, or each piece thereof, may be configured to deliver, elute, or otherwise release the therapeutic composition. In some embodiments, the porous material may be configured to receive and deliver a therapeutic composition to one or more target cardiac tissues either adjacent to or removed from the clip.

Depending on the embodiment, the one or more pieces of porous material may be attached to the first and/or second arm of a clip in any appropriate manner. For example, as shown in the cross-sectional view of FIG. 2C, an arm of an atrial clip may include a groove to facilitate attachment of the porous material to the arm. In the embodiment shown, the first arm 202 may include a first groove 208 and the second arm 204 may include a second groove 218 both of which may extend along at least a portion of, or entire, length of the arms. As seen in the figure, the porous material 214 and 216 may be disposed within and extend out from the associated groove. The porous material may also extend along at least a portion of, an entire, or past a length of the corresponding groove it is partially disposed in. The porous material may be attached to the corresponding arm within the groove using an any appropriate attachment methods including an adhesive. Depending on the specific embodiment, the adhesive may be a biocompatible adhesive such as cyanoacrylate, although it will be appreciated that any appropriate adhesive may be used as the disclosure is not limited in this respect. Additionally or alternatively, a porous material 214 or 216 may be secured within the corresponding groove 208 or 218 mechanically. For example, the first piece of porous material 214 may be attached to the first arm 202 within the first groove 208 using clips, crimps, clamps, snaps, mechanical interlocking features, or other fasteners.

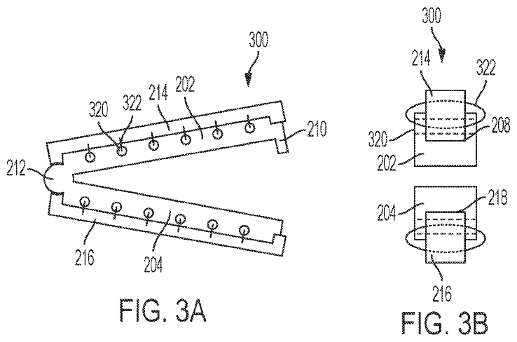

FIGS. 3A-3B illustrate an exemplary embodiment of an atrial clip 300 in which the porous materials 214 and 216 may be attached to the first arm 202 and second arm 204 by stitching the porous material to the arm using sutures 322. In some embodiments, each suture 322 may pass through a through hole 320 formed in a corresponding arm of the atrial clip 300. Additionally, each suture 322 may pass through the corresponding piece of porous material, in order to attach the porous material to the atrial clip. In the embodiment shown, the porous materials 214 and 216 may be attached to the corresponding first and second arms 202 and 204 of an atrial clip 300 using a plurality of sutures 322 and a plurality of through holes 320. As illustrated in FIGS. 3A-3B, sutures 322 may be used in conjunction with grooves 208 and 218 similar to those described above, although it will be appreciated that sutures may be used without a groove as well. As illustrated, in embodiments which use sutures and a groove, each suture 322 may pass through the first piece of porous material 214 twice. This may provide a more secure attachment between the porous material and the atrial clip as compared to an embodiment in which each suture passes through the porous material only once, as may be the case when the atrial clip does not include a groove.

A suture may be formed from any appropriate suture material. Depending on the specific embodiment, the suture may be formed from a biocompatible suture material such as polypropylene, although it will be appreciated that any appropriate suture material may be used as the disclosure is not limited in this respect

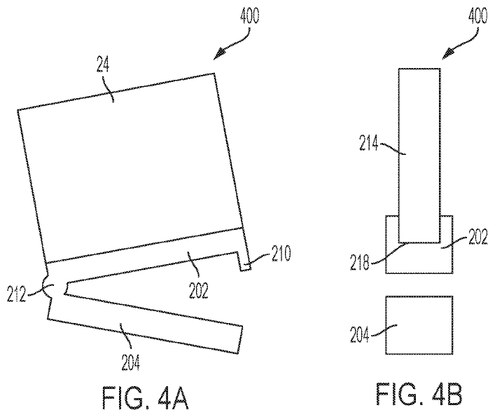

FIGS. 4A-4B depict an embodiment of an atrial clip having only a single piece of porous material attached to a correspond clip. In this embodiment, a piece of porous material 214 may be attached to a first arm 202 of an atrial clip 400 in the same or similar fashion to the embodiment of FIGS. 2A-2C. It may be noted that the piece of porous material 214 is illustrated in FIGS. 4A-7B as being larger than the first piece of porous material 214 depicted in FIGS. 2A-3B, in comparison to a size of an atrial clip as depicted. Dimensions and proportions such as those illustrated by the piece of porous material 214 may allow the porous material to be relatively flexible such that it may be draped over and/or otherwise conform to the shape of a structure the clip is attached to during a surgical procedure. However, it will be appreciated that an embodiment having one piece of porous material need not have a porous material of any particular size. Similarly, the porous material of an embodiment having multiple pieces of porous material need not have any particular size, as the present disclosure is not limited with respect to the size of the porous material. Additionally, in some embodiments, the porous material may be disposed on an arm as a thick porous coating having a suitable thickness and porosity to provide a desire loading of a therapeutic composition absorbed therein.

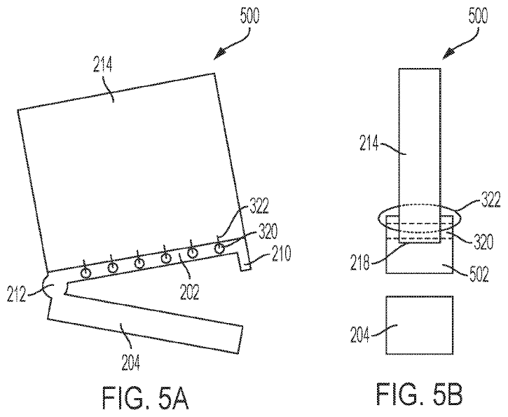

A first groove 218 may be optionally included in the first arm 202 to facilitate attachment of the piece porous material 214 to the first arm 202 where the porous material may be disposed partially in and extend out from the groove in a manner similar to that described above. Similar to the above, the porous material may be attached to the groove with an adhesive or mechanical fastening mechanism as previously described. For example, FIGS. 5A-5B depict an exemplary embodiment in which the piece of porous material 214 may be attached to the first arm 202 by stitching the porous material to the arm using sutures 322, through holes 320, and (optionally) a groove 218. The use of sutures 320 is particularly described in relation to FIGS. 3A-3B above.

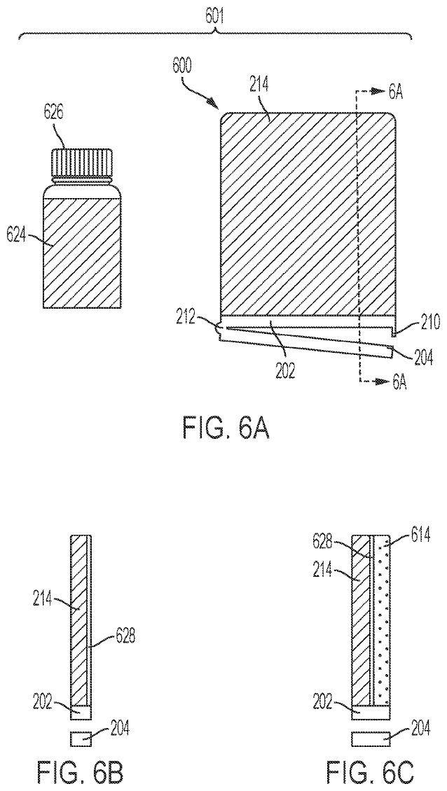

As shown in FIG. 6A, the devices and therapeutic compositions described herein can be provided as a kit 600. The kit 600 can include one or more atrial clips 600 for delivering a therapeutic composition. A porous material 214 of the atrial clips 600 may come in many different shapes and sizes or may be cut to size during a procedure, depending on need. A quantity of one or more therapeutic compositions 624 may also be provided. Such compositions may be provided in a liquid form, e.g., as a pure liquid, suspension, solution or emulsion, or as a crystalline or powdered solid, or even in gaseous form. Such substance(s) can be provided in a dispensing bottle or container 626. The therapeutic composition can be provided in such containers in pre-measured volumes or doses.

In some embodiments, a surgeon or medical professional may open a kit 600 and remove the atrial clip 600 and the container 626 holding the therapeutic composition 624. The therapeutic composition 624 may be applied to the porous material 214 in the operating room prior to a procedure. For example, the porous material 214 may be soaked in the therapeutic composition 624 when the therapeutic composition is supplied in liquid form. However, embodiments in which the therapeutic composition is already preloaded into the porous material and is ready for use by a surgeon or other medical professional are also contemplated.

In some embodiments, the therapeutic composition 624 may include a therapeutic composition for preventing or treating POAF after heart surgery. For example, the therapeutic composition may include amiodarone. In some embodiments, the therapeutic composition may include amiodarone combined with other small molecules such as lidocaine, magnesium, beta-blockers, statins, and/or any other appropriate therapeutic composition. These components may have synergistic effects when used in combination by affecting both potassium and calcium channels in the myocardium, helping to prevent risk of POAF. Of course, other types of therapeutic compositions intended to prevent or treat postoperative atrial fibrillation and/or therapeutic compositions for preventing or treating other types of conditions may be used with the materials and devices disclosed herein as the disclosure is not so limited.

In some embodiments, a therapeutic composition may be applied to a porous material in the operating room and/or preloaded onto the porous material 214 during manufacturing or prior to being assembled in a kit 600. In an atrial appendage closure procedure for example, a therapeutic composition 624 of amiodarone optionally combined with one or more of magnesium, lidocaine, beta-blockers, and/or statins may be supplied in a container, such as a vial 626, of a kit 600, and the porous material 214 may be soaked in the therapeutic composition 624 in the operating room prior to the procedure. In some embodiments, amiodarone, magnesium, lidocaine, and/or any other appropriate therapeutic composition may be pre-loaded onto the porous material 214. For example, magnesium may be preloaded onto the porous material 214 and/or amiodarone may be preloaded into the porous material via various methods, such as precipitation and/or crystallization. When porous materials 214 are pre-loaded with amiodarone, additional amiodarone may be added in liquid formulation in the operating room, provided as part of the therapeutic composition 624. The combination of precipitated and liquid amiodarone can increase the concentration of amiodarone delivered to the heart and also make the delivery bimodal with faster release via liquid and slower release via the crystallized amiodarone. Embodiments in which this two-phase delivery concept is used for other therapeutic compositions are also contemplated in which a first therapeutic composition may be provided in a liquid form that is applied just prior to implantation and a solid form of either the same therapeutic composition and/or a second different therapeutic composition may be preloaded within the porous material. Similar to the above, the first therapeutic composition loaded in the liquid form may undergo a first elution rate and the solid therapeutic composition may undergo a second slower elution rate.

As noted above, in some embodiments, it may be desirable to provide directionality for a therapeutic composition eluted from a device as disclosed herein. One such embodiment is shown in FIG. 6B, which illustrates a cross section of one embodiment of the atrial clip of FIG. 6A taken along line A-A. In the embodiment of FIG. 6B, a barrier layer 628 is disposed on, or in, a first surface of the porous material 214 attached to one or more of arms 202 and 204 of a clip such that the therapeutic composition may be eluted exclusively or primarily from an opposing second surface of the porous material 214. Correspondingly, the barrier may reduce, and in some embodiments, substantially prevent, the elution of the therapeutic composition from the surface of the porous material on which the barrier layer is positioned.

In yet another embodiment, it may be desirable to provide different therapeutic compositions to different structures adjacent to opposing surfaces of a porous material 214. One such embodiment is shown in FIG. 6C, which illustrates a cross section of another embodiment of the atrial clip of FIG. 6A taken along line A-A. In the embodiment of FIG. 6C, a first porous material 214 and a barrier layer 628 disposed thereon may be used to apply a first therapeutic composition to a first structure, such as the heart. A second porous material 614 may be disposed on a surface of the barrier layer 628 opposite from the first porous material 214. The second porous material 614 may be loaded with a second therapeutic composition such that the second composition is eluted from the exterior surface of the second porous material 614 but is substantially prevented from diffusing through the barrier layer. This second porous material may be attached to one or more of the arms 202 and 204 of a clip similar to the first porous material. For example, lidocaine or another pain reliever might be included in the second porous material. In such an embodiment, a first therapeutic composition may be delivered to the heart from a heart oriented surface of the device while the other therapeutic composition, such as the disclosed pain reliever, may be delivered to the sternum from a sternal oriented surface of the device. Thus, the disclosed devices may be used to deliver different therapeutic compositions to different anatomical structures located on opposite sides of a device.

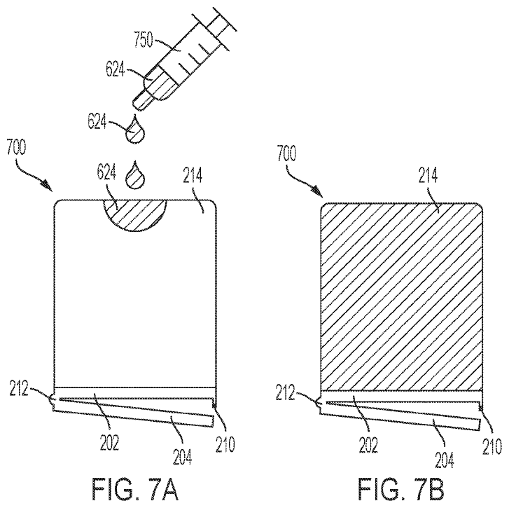

FIG. 7A shows an embodiment of a porous material 214 during a soaking process in a therapeutic composition 624. FIG. 7B shows a porous material 214 fully soaked in the therapeutic composition 624. As shown in FIG. 7A, a medical professional may use a syringe 750 or other applicator to apply the therapeutic composition 624 to the porous material 214. A sufficient amount of therapeutic composition 624 may be applied to the porous material 214 until the porous material is fully saturated, as shown in FIG. 7B. Embodiments in which the porous material 214 is simply immersed in a solution are also contemplated as the disclosure is not limited to how the porous material is exposed to the therapeutic composition. After a sufficient duration to permit the desired amount of therapeutic composition to be absorbed into the porous material, the porous material may then be used for a desired surgical procedure.

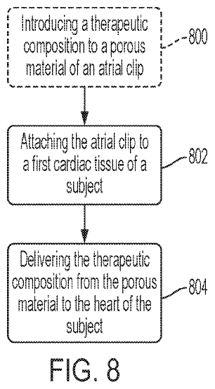

A method of delivering a therapeutic composition to a heart is shown in FIG. 8. In step 800, a therapeutic composition is introduced to a porous material. Step 800 may be carried out in an operating room during a procedure. In some embodiments, as discussed above, the porous material may be soaked in the therapeutic composition immediately prior to the atrial clip being attached to a heart tissue of a subject. Alternatively, step 800 may be performed during manufacturing of the porous material or the atrial clip, or at some time prior to the surgical procedure, such that the porous material is preloaded with therapeutic agents at the time of the procedure. A porous material pre-loaded with a therapeutic composition may or may not be soaked in additional therapeutic agents at the time of surgery. Thus, step 800 is an optional step in the described method, as denoted by the dashed lines enclosing step 800.

In step 802, the atrial clip is attached to a first cardiac tissue of a subject. For example, the atrial clip may be attached to an atrial appendage. However, embodiments in which a clip is attached to other cardiac tissues are also contemplated. In step 804, the therapeutic composition in the porous material is delivered from the porous material to the heart of the subject. Specifically, the porous material may act as a reservoir for the therapeutic composition. Once the porous material is attached to the heart tissue via a clip, the therapeutic composition may be delivered from the porous material to the cardiac tissue.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A clip comprising:
a first arm;
a second arm;
a hinge rotatably coupling the first arm and the second arm, wherein the first arm and the second arm are selectively moveable between an open configuration and a closed configuration, and wherein the first arm and the second arm are configured to engage with and clamp cardiac tissue therebetween in the closed configuration;
a lock configured to retain the first arm and the second arm in the closed configuration; and
at least one porous material attached to at least one of the first arm and the second arm, wherein the at least one porous material is configured to absorb and elute a therapeutic composition.

2. The clip of claim 1, wherein the first arm and second arm are configured to engage with and clamp an atrial appendage therebetween in the closed configuration.

3. The clip of claim 1, wherein the clip is an atrial clip.

4. The clip of claim 1, wherein the first arm includes a first groove and wherein a first porous material of the at least one porous material is disposed partially within and is attached to the first groove.

5. The clip of claim 1, further comprising a second porous material of the at least one piece of porous material, the second porous material being attached to the second arm.

6. The clip of claim 5, wherein the second arm includes a second groove, and the second porous material is disposed partially within and is attached to the second groove.

7. The clip of claim 1, wherein the at least one porous material is attached to the first arm by at least one selected from the group consisting of: stitches, clamps, mechanically interlocking features, fasteners, and adhesives.

8. The clip of claim 1, wherein the at least one porous material comprises a biopolymer scaffold material.

9. The clip of claim 8, wherein the biopolymer scaffold material comprises a decellularized bovine extracellular matrix.

10. The clip of claim 9, wherein the decellularized bovine extracellular matrix comprises fetal or neonatal tissue.

11. The clip of claim 1, wherein the at least one porous material comprises at least one selected from permanent or degradable synthetic or natural polymers, biopolymer scaffold materials, and collagen foams.

12. The clip of claim 1, wherein the at least one porous material has a thickness of equal to or greater than approximately 0.2 mm and less than or equal to approximately 6 mm.

13. The clip of claim 1, wherein the at least one porous material has a surface area of equal to or greater than approximately 1 cm² and less than or equal to approximately 200 cm².

14. The clip of claim 1, wherein the at least one porous material has a porosity of equal to or greater than approximately 20% and less than or equal to approximately 80%.

15. The clip of claim 1, wherein the at least one porous material has a tensile strength of equal to or greater than approximately 3 MPa and less than or equal to approximately 60 MPa.

16. The clip of claim 1, wherein the at least one porous material has a suture retention strength of equal to or greater than approximately 10 N and less than or equal to approximately 500 N.

17. The clip of claim 1, wherein the at least one porous material has an areal absorption capacity of liquid of equal to or greater than approximately 0.1 ml/cm² and less than or equal to approximately 1 ml/cm².

18. The clip of claim 1, wherein the therapeutic composition comprises at least one selected from the group consisting of: amiodarone, lidocaine, magnesium, salicylic acid, beta-blockers, statins, and antibiotics.

19. The clip of claim 18, wherein the at least one porous material comprises 1 mg to 3000 mg of amiodarone.

20. The clip of claim 1, further comprising the therapeutic composition disposed in the at least one porous material.

21. The clip of claim 20, wherein the therapeutic composition comprises at least one selected from the group consisting of: amiodarone, lidocaine, magnesium, salicylic acid, beta-blockers, statins, and antibiotics.

22. A method of delivering a therapeutic composition to a heart of a subject, the method comprising:
attaching a clip to a first cardiac tissue, the clip including a porous material containing the therapeutic composition;
delivering the therapeutic composition from the porous material to the heart of the subject.

23. The method of claim 22, wherein delivering the therapeutic composition to the heart of the subject comprises delivering the therapeutic composition to an epicardial tissue, a pericardial tissue, and/or a myocardial tissue of the heart.

24. The method of claim 22, wherein the porous material comprises a biopolymer scaffold material.

25. The method of claim 24, wherein the biopolymer scaffold material comprises a decellularized bovine extracellular matrix.

26. The method of claim 22, wherein the porous material comprises at least one selected from permanent or degradable synthetic or natural polymers, biopolymer scaffold materials, and collagen foams.

27. The method of claim 22, wherein attaching the clip to the first cardiac tissue comprises attaching the clip to an atrial appendage of the heart.

28. The method of claim 27, wherein attaching the clip to the atrial appendage of the heart comprises attaching the atrial clip to the atrial appendage of the heart to occlude blood flow to the atrial appendage.

29. The method of claim 22, wherein the clip is an atrial clip.

30. The method of claim 22, further comprising introducing the therapeutic composition to the porous material.

31. The method of claim 22, further comprising introducing the therapeutic composition to the porous material via soaking.

32. The method of claim 22, wherein the therapeutic composition is configured to prevent and/or treat postoperative atrial fibrillation.

33. The method of claim 32, wherein the therapeutic composition comprises at least one selected from the group of amiodarone, lidocaine, magnesium, salicylic acid, beta-blockers, statins, and antibiotics.

34. The method of claim 22, further comprising confining the therapeutic composition to within the heart of the subject.
